(19) United States
(12) Patent Application Publication
(10) Pub. No.: US 2021/0106777 A1
(43) Pub. Date: Apr. 15, 2021

(54) Device For Assisting With The Use Of A Device For Dispensing A Liquid Product

(71) Applicant: Nemera La Verpillière, LA VERPILLIERE (FR)

(72) Inventors: Thierry Decock, Lyon (FR); Loïck Cassagne, Saint-Alban-de-Roche (FR); Pierre Pintus, CRACHIER (FR)

(21) Appl. No.: 16/968,054

(22) PCT Filed: Feb. 5, 2019
(86) PCT No.: PCT/EP2019/052778

§ 371 Date: Dec. 24, 2020

(30) Foreign Application Priority Data
Feb. 6, 2018 (FR) 18/50991

(51) Int. Cl. A61M 15/00

(52) U.S. Cl. CPC A61M 15/0068; A61M 2205/3393; A61M 2205/332; A61M 2205/3306

(57) ABSTRACT

The invention relates to an assistance device (10) for assisting in the use of an axially activated dispensing device (12) for dispensing a product contained in a tank (14), the dispensing being activated by the user following pressure exerted by the user on a bearing area (20) mounted so as to be able to move with respect to the tank (14) between a rest position and an activated dispensing position, the assistance device (10) comprising:
means for connecting it to the dispensing device (12),
means for quantifying a physical value linked to the force exerted by the user on the bearing area (20) between the rest position and the activated position,
an information processing system for processing the quantified physical value and providing information on the dispensed product dose.

The invention also relates to a method for monitoring the dispensing of a complete dose during the activation of a product dispensing device (12), by means of such an assistance device (10).

The invention relates to a device for assisting in the use of an axially activated dispensing device and a method for monitoring the dispensing of a complete dose during the activation of a product dispensing device.

When a product is dispensed, it is often interesting to know the amount of product dispensed. This is particularly true for the administration of medical products, for which the amount of medication administered must be precisely controlled according to the prescription. An underdose or overdose of medication must be avoided to preserve the patient's health, and also, knowing the amount of product dispensed, the amount of product remaining in the dispensing device can be calculated.

Document WO 2017/178767 describes a device for dispensing product by inhalation. The dispensing device is in particular capable of detecting the displacements of the tank containing the product by means of a contact sensor. When the tank moves to dispense a dose, in fact, it comes into contact with the contact sensor which is arranged on its path. The administration of a dose can therefore be deduced and the number of doses dispensed calculated. However, this device does not provide information relating to the amount of a dispensed dose, in particular whether the dispensed dose was complete or incomplete.

The invention aims in particular to provide a device for detecting an activation dispensing a complete product dose.

Thus, the invention relates to a device for assisting in the use of an axially activated dispensing device for dispensing a product contained in a tank, the dispensing being activated by the user following a pressure exerted by the user on a bearing area mounted so as to be able to move with respect to the tank between a rest position and an activated product dispensing position, the assistance device comprising:
means for connecting to the dispensing device,
means for quantifying a physical value linked to the force exerted by the user on the bearing area between the rest position and the activated position,
an information processing system for processing the quantified physical value and providing information on the dispensed product dose.

Thus, it is proposed to quantify a physical value linked to the force exerted on the bearing area between the rest position and the activated position. It will then be possible to determine whether or not a dose has been dispensed, and even more advantageously, whether or not the dispensed dose is complete, i.e. whether the amount of product dispensed corresponds to a predetermined fixed amount of product or only part of this amount. Some doses may in fact vary depending on different physical parameters related to the conditions under which the dispensing device is activated. Incorrect use of the device may lead to the administration of an incomplete dose, i.e. a dose quantitatively less than the predetermined amount. However, if this incomplete dose is considered to be complete, the dosage may not be respected.

An “incomplete” administered dose means a dose which has been incompletely expelled from the dispensing device, or a dose which has been completely expelled, but incorrectly, in other words poorly dispensed, due to incorrect use of the dispensing device which may, for example, result in expulsion of the liquid product which is too slow or inefficient, with a poor spatial distribution of the drops of a spray or a poorly adapted spray profile.

In addition, by quantifying the force exerted by the user on the bearing area, the residual number of doses in the tank can be determined. In addition, the possibility of obtaining an incorrect value of this number, caused by dispensing an incomplete dose considered to be complete, can be avoided. This information is likely to be of interest to users, by allowing them to know the amount remaining in the tank, which is generally unknown from the outside. The information can also be used for other types of analysis or processing to obtain additional information.

Note that the “information on the dispensed product dose” can take numerous forms. It may be qualitative information, such as a “dose not dispensed” or “dose dispensed”, and/or it may also be, when the dose is dispensed, quantitative information on this dose. Furthermore, regardless of whether the information on the dispensed product dose is qualitative and/or quantitative, the processing system can advantageously provide quantitative information using the quantified physical value by combining it, or not, with other information such as the physical or chemical properties of the product, the geometry of the dispensing orifice, or tables of predefined values. This quantitative information comprises, for example, the exact amount of the dose, for example a dose volume, a drop size or even quantitative information relating to the spatial distribution of a spray or a drop.

It is understood that an “axially activated dispensing device” comprises any type of dispensing device which requires the application of a force by the user in the product dispensing axis to dispense product. The dispensing device may be a device for dispensing a product in various forms, in particular in the form of a spray or in the form of drops, or a device for dispensing an aerosol. Furthermore, the dispensing device may be a nasal, buccal, auricular, ophthalmic or dermal device.

The bearing area corresponds to an area mounted so as to be able to move with respect to the tank between the rest position and the activated position. Preferably, users press directly on the bearing area, however they can also press indirectly thereon. Furthermore, the bearing area can be carried by the assistance device and/or the dispensing device. The case where the bearing area is carried by the assistance device is advantageous since this bearing area of the assistance device also increases the users gripping area compared with that of the dispensing device alone, which is particularly advantageous especially for users with neuromuscular diseases.

Moreover, it is understood that the “means for quantifying a physical value linked to the force exerted by the user on the bearing area between the rest position and the activated position” are configured to be arranged directly or indirectly on the bearing area of the assistance device. They can be arranged near this bearing area, or at a certain distance from it, which may be the case when these means comprise a force-sensing resistor arranged under the tank.

The assistance device may further comprise one or more of the following characteristics, taken alone or in combination.
The means for quantifying a physical value comprise means for measuring the distance of displacement of the bearing area between the rest position and the activated position. The displacement of the bearing area between the two positions is in fact indicative of the dispensing of a dose. By measuring the distance of this displacement and by providing this information to the processing system, the processing system can determine whether the dispensed dose has been dispensed and whether or not it is complete, for example by comparing the distance measured with a predetermined distance threshold. It is understood that the distance of displacement of the bearing area between its rest and activated positions is a relative distance of displacement with respect to the tank and which corresponds to the distance of displacement of the tank with respect to the bearing area when the dispensing device is activated. Thus, “means for measuring the distance of displacement of the bearing area between the rest position and the activated position” refers to means for measuring the distance of displacement of the bearing area directly, or means for measuring the distance of displacement of the bearing area indirectly by measuring the distance of displacement of the tank with respect to the bearing area.
The means for quantifying a physical value comprise means for measuring the duration of displacement of the bearing area with respect to the tank, the information processing system being capable of providing information on the speed of displacement of the bearing area with respect to the tank. It is thus proposed to obtain the speed of displacement of the bearing area between its two positions, since this parameter affects the amount of the dispensed dose. The information processing system can therefore advantageously deduce quantitative information on the dispensed dose, for example by comparing the deduced speed with predetermined speed curves/charts. As for the means for measuring the distance of displacement of the bearing area, the means for measuring the duration of displacement of the bearing area can measure this duration directly or indirectly by using the duration of displacement of the tank with respect to the bearing area.
The means for measuring the distance and/or the duration of displacement of the bearing area comprise optical means configured to measure the distance of displacement, and preferably the duration of displacement, by the transmission and reception of an optical signal between the bearing area and the tank. The optical means can take various configurations. The receiver and the transmitter may both be arranged on the bearing area (respectively on an element fixedly mounted with respect to the tank, for example on the connection means), the optical signal is then reflected on the tank (respectively on the bearing area) and the distance and/or the duration of displacement of the bearing area is deduced by measuring the duration between the transmission and the reception of the optical signal, the speed of the optical signal being known. Alternatively, the receiver is arranged on the bearing area (respectively on an element fixedly mounted with respect to the tank) and the transmitter on an element fixedly mounted with respect to the tank (respectively to the bearing area). In this configuration, by comparing the duration between the transmission and the reception of the optical signal with the reference duration without displacement of the bearing area, the distance and the speed of displacement of the bearing area can be obtained. An “optical signal” means any electromagnetic wave, belonging to the visible or non-visible spectrum. The optical means are generally means capable of transmitting, receiving and/or reflecting such a wave.
The means for quantifying a physical value comprise means for measuring the pressure exerted on the bearing area, in order to provide information on the magnitude and/or duration of the pressure exerted by the user on the bearing area. A pressure of sufficient magnitude and/or duration exerted on the bearing area in the dispensing direction will displace the bearing area from its rest position to its activated position and thereby trigger the dispensing of a dose. Thus, to determine whether the dispensed dose is complete, the magnitude and/or the duration of the pressure exerted are measured, since these parameters affect the amount of product dispensed. It is understood that the means for measuring the pressure exerted on the bearing area can be fixedly mounted with respect to the tank. When the user exerts a pressure on the bearing area, in fact, this pressure is transmitted at least partially to the tank, so that means for measuring the pressure connected to the tank receive a pressure variation indicative of an activation of the dispensing device.
The means for quantifying a physical value comprise means for measuring the acceleration of the bearing area. Advantageously, the means for measuring the acceleration comprise an accelerometer arranged directly on the bearing area or on an element fixedly mounted with respect to the bearing area. As for the means for measuring the distance and/or the duration of displacement of the bearing area, the means for measuring the acceleration of the bearing area can measure this acceleration directly or indirectly by using the duration of displacement of the tank with respect to the bearing area. In this second case, the accelerometer can be arranged either directly on the tank or on an element fixedly mounted with respect to the tank, for example on a main body housing the tank. The combination of the measurement of pressure using the means described above and the measurement of acceleration over time is particularly advantageous since this information can be used to determine whether the dispensed dose is complete, while avoiding false positives. By taking the measurement using an accelerometer alone, it is not necessarily possible to know whether the measurement obtained is due to a relative acceleration between the bearing area and the tank or to an acceleration of the dispensing device taken as a whole. It is therefore interesting to combine this measurement with that taken using the means for measuring the pressure to ensure that the acceleration measured is relative between the bearing area and the tank. Moreover, we see that an incomplete dose may be dispensed if a pressure of satisfactory magnitude is applied for an insufficient period of time, or if a pressure of unsatisfactory magnitude is applied for a sufficient period of time. The combined measurement of the two parameters can be used to deduce whether a complete dose has been dispensed and then deduce the remaining number of doses in the dispensing device.
An example of an element fixedly mounted with respect to the bearing area is a dispensing end piece carried by the dispensing device connected to the assistance device. It is understood that the dispensing end piece is a part of the dispensing device carrying the dispensing orifice and which is mounted so as to be able to move with respect to the tank of the dispensing device, its displacement activating the dispensing of product when the user presses on the bearing area. Note that the means for measuring the acceleration may comprise an electronic gyroscope instead of the accelerometer.
The means for quantifying a physical value comprise means for measuring the relative acceleration between the bearing area and the tank, preferably two accelerometers, a first accelerometer being fixedly mounted with respect to the bearing area and a second accelerometer being fixedly mounted with respect to the tank, the information processing system being capable of processing the acceleration information provided by the two accelerometers in order to deduce the relative acceleration between the bearing area and the tank. By using two accelerometers, the relative acceleration of the bearing area with respect to the tank can be measured, thereby avoiding false positives. For example, when the bearing area and the tank move synchronously but are stationary relative to each other, the two accelerometers provide the same measurements, and a false positive can therefore be identified (i.e. the first accelerometer indicates a displacement but the second accelerometer indicates the same displacement, this displacement is therefore not indicative of an activation), without the need for means for measuring the pressure as described previously. By measuring the relative acceleration between the bearing area of the assistance device and the tank of the dispensing device, the speed of the bearing area with respect to the tank at the time of its activation can be deduced, and information used to deduce the amount of the dispensed dose can be provided.
The assistance device comprises means for measuring the weight of the dispensing device connected to the assistance device, configured to provide information on the amount of product in the tank. The weight measurement provides direct information on the remaining amount of product, the relative density of the product being a priori known, for example being pre-recorded or read on the dispensing device. Also, by comparing the weight measurement with the information provided by the means for quantifying a physical value, accurate quantitative information is provided on the dispensed dose and the remaining number of doses. In addition, correct dispensing of a dose can be checked. In particular, if no weight change is detected, the number of doses will not be decreased. Advantageously, the means for measuring the weight comprise a weight sensor, for example of the Force Sensing Resistor (FSR) type, under and/or above the tank. An example of a suitable weight sensor is the force-sensing resistor manufactured by Interlink Electronics capable of measuring forces within the range 20 gf to 2.0 kgf.
The processing system is configured to provide quantitative information on the dispensed product dose, for example a dose volume, a drop size or even quantitative information relating to the spatial distribution of a spray or drops. To obtain this information, the processing system uses the information on the distance and/or the speed of displacement of the bearing area between the rest position and the activated position, and/or the relative acceleration between the bearing area and the tank, and/or the weight of the dispensing device connected to the assistance device.
The processing system comprises means for reading information shown by the dispensing device. The processing system can then access various pieces of information on the product contained in the dispensing device or on the dispensing device itself, not requiring manual configuration of the assistance device. These pieces of information are for example the number of theoretical doses contained in the tank, the theoretical amount of a drop, the physical or chemical properties of the product such as the product viscosity, the dimensions of the dispensing end piece, tables of predetermined values, or calculation tools. Some of these pieces of information may be useful for processing the information provided by the various means, for example the means for quantifying a physical value. The dispensing device may comprise information such as digital chips, magnetic tapes, bar codes, or any other type of medium storing information which can be read electronically.
The processing system comprises means for storing a variable value on the amount of product remaining in the tank. The variable value is for example used to deduce the amount of product dispensed and control the amount of product remaining in the tank. It is updated when product is dispensed. By storing these values, the change in the number of doses dispensed and/or remaining can be monitored over time. Advantageously, the storage means can store other values and information useful for the processing system and/or the user.
The assistance device comprises means for indicating the information provided by the processing system, for example visual means, audible means and/or tactile means. These means can be used to indicate to the user information on the effective dispensing of a dose, the dispensing of a complete or incomplete dose, the amount of product dispensed and/or remaining and/or other information on the product (for example read by the reading means). The display means may comprise a screen to display information alphanumerically and/or light signals, for example of different colour or shape.
The connection means are configured to be connected to a dispensing device comprising a pump. A pump-type dispensing device generally corresponds to an aerosol-free device. It is particularly advantageous to be able to measure the pressure exerted by the user on the bearing area and/or the distance of displacement and/or the speed of displacement of the bearing area since it can have a greater impact on the dispensed dose than with an aerosol. With a pump, in fact, product is dispensed by the users action alone, there is no pressure to assist with the dispensing.
The assistance device is configured to be connected to a dispensing device comprising an aerosol dispensing valve.
The assistance device comprises means for activating and/or waking up the means for quantifying a physical value, comprising for example means for detecting the pressure exerted on the bearing area. Since the user must exert a pressure to move the bearing area and dispense a product dose, it may be decided to wake up components above a certain pressure, which may be a minimum pressure to dispense a product dose or a pressure less than this minimum pressure. Thus, each activation can be detected by quantifying the one or more physical values from this time. When such a pressure is not detected, it is advantageous to use fewer components to save energy and simplify the data. “Waking up components” means switching on electronic components or taking them out of a power-saving mode.
The bearing area comprises two bearing surfaces arranged on each side of a dispensing orifice which is carried by the dispensing device connected to the assistance device, for example a bearing collar configured to receive a users index finger and middle finger. The bearing area can be made in one piece with a dispensing end piece in which the dispensing orifice is formed, or attached to this dispensing end piece, in particular if the bearing area is carried by the assistance device.

The invention also relates to a method for monitoring the dispensing of a complete dose during the activation of a product dispensing device, by means of an assistance device as described previously, comprising the following steps:
quantifying a physical value linked to the force exerted by the user on the bearing area between the rest position and the activated position,
qualifying the dispensed dose using the quantified physical value.

The force exerted on the bearing area between the rest position and the activated position, generated by the application of a force by the user on the bearing area, is in fact indicative of the dispensing of a dose. By quantifying a physical value linked to this force, we obtain firstly information on whether or not a dose has actually been dispensed, and secondly qualitative (complete or incomplete) and/or quantitative information on the dose dispensed (such as amount dispensed, spatial distribution of the drops, drop size, etc.).

The monitoring method may further comprise one or more of the following characteristics, taken alone or in combination.
The step of quantifying a physical value comprises a step of measuring the distance of displacement of the bearing area between the rest position and the activated position. The distance of displacement measured may provide at least a piece of qualitative information on the dispensed dose, i.e. whether or not the dispensed dose is complete.
The step of quantifying a physical value comprises a step of measuring the duration of displacement of the bearing area between the rest position and the activated position, which can be used to deduce the speed of displacement with respect to the tank. When the distance of activation of a dispensing device is known, measurement of the duration of displacement alone can be used to deduce the speed of displacement.
The step of measuring the distance, and preferably the duration, of displacement of the bearing area comprises a step of transmitting and a step of receiving an optical signal between the bearing area and the tank, preferably comprising a step of reflecting the optical signal on the tank or on the bearing area. In one example, the optical signal transmitted from the bearing area (respectively from the tank) to the tank (respectively to the bearing area) is reflected on the tank (respectively on the bearing area) to be received at the bearing area (respectively at the tank). In this case, the distance travelled by the optical signal, for example an infrared ray, and its travel time, can be about twice the distance and about twice the travel time, respectively, between the bearing area and the tank. In another example, the optical signal transmitted from the bearing area (respectively from the tank) is received directly at the tank (respectively at the bearing area). In this case, the distance travelled by the optical signal and the travel time reflect the distance travelled between the bearing area and the tank. In both cases, a variation in the distance and the travel time of the optical signal reflects a variation in the distance (or twice the distance) between the bearing area and the tank and can therefore be used to measure the displacement of the bearing area with respect to the tank over time.
When qualifying the dispensed dose, the dispensed dose is considered to be complete if the distance of displacement of the bearing area is greater than a predefined threshold, the distance of displacement preferably being travelled in a duration less than a predefined threshold. Alternatively, the dispensed dose can be considered to be complete if the distance of displacement is travelled at a speed greater than a predefined threshold. For some dispensing devices, a complete dose is dispensed by the displacement of the bearing area over a fixed distance. In this case, the predefined threshold corresponds to this fixed distance and the term “greater than” is to be understood as “greater than or equal to”.
The step of quantifying a physical value comprises a step of measuring the pressure exerted on the bearing area.
The dispensed dose is considered to be complete if the magnitude of the pressure exerted exceeds a predefined threshold, preferably for a duration greater than a predefined threshold. To dispense a complete dose, it is often preferable for the user to exert a sufficient pressure, preferably for a sufficient duration, on the bearing area.
The step of quantifying a physical value comprises a step of measuring the acceleration of the bearing area. To avoid false positives, in particular to distinguish between the case where the entire dispensing device moves and that where only the bearing area moves with respect to the tank, it is interesting to combine the measurement of the acceleration of the bearing area with the measurement of the magnitude and/or the duration of the pressure exerted on the bearing area.
The step of quantifying a physical value comprises a step of measuring the acceleration of the bearing area and simultaneously the acceleration of the tank, and a step of determining the relative acceleration of the bearing area with respect to the tank. By measuring the two accelerations, we obtain firstly the relative acceleration of the bearing area with respect to the tank by subtracting the two accelerations, and secondly false positives are avoided by taking into account only the displacements of the bearing area with respect to the tank.
When qualifying the dispensed dose, the dispensed dose is considered to be complete if the relative acceleration measured is greater than a predefined threshold, then preferably less than a predefined threshold. A complete dose is generally dispensed when the user exerts a sufficient acceleration on the bearing area with respect to the tank, but preferably an acceleration which is low enough to minimise the risks of incorrect operation.
The amount of product dispensed is considered to correspond to a complete dose if the integral of the acceleration for a predefined duration comprises the succession of a positive value and a negative value, each within a predefined time interval. In other words, the curve of acceleration measurement against time includes a set of positive values followed by a set of negative values, which corresponds to a speed which increases and then decreases. The acceleration measurement curve is thus divided into three zones.
The step of quantifying a physical value comprises a step of measuring the weight of the dispensing device connected to the assistance device.
During the step of qualifying the dispensed dose, the dispensed product dose is quantified. In particular, the dispensed product dose is quantified using the information on the distance and/or the duration of displacement of the bearing area with respect to the tank, and/or the relative acceleration between the bearing area and the tank, and/or the weight of the dispensing device connected to the assistance device.
A sudden variation in the acceleration is detected and, when qualifying the dispensed dose, the dispensed dose is considered to be complete if the intensity of these variations is greater than a predetermined threshold. The sudden variation in the acceleration results in a variation in the second derivative of the curve of acceleration against time.
Recording of the information on the magnitude of the pressure exerted by the user and/or on the acceleration of the bearing area starts if the magnitude of the pressure exerted exceeds a predefined threshold.

Lastly, the invention relates to a kit for dispensing a product, comprising a device for dispensing the product and an assistance device as described previously. Preferably, the assistance device and the dispensing device are separate devices, attached to each other and removable, but they could form a single unit and be made of the same material.

We will now describe particular embodiments of the invention given as non-limiting examples referring to the attached figures, on which:

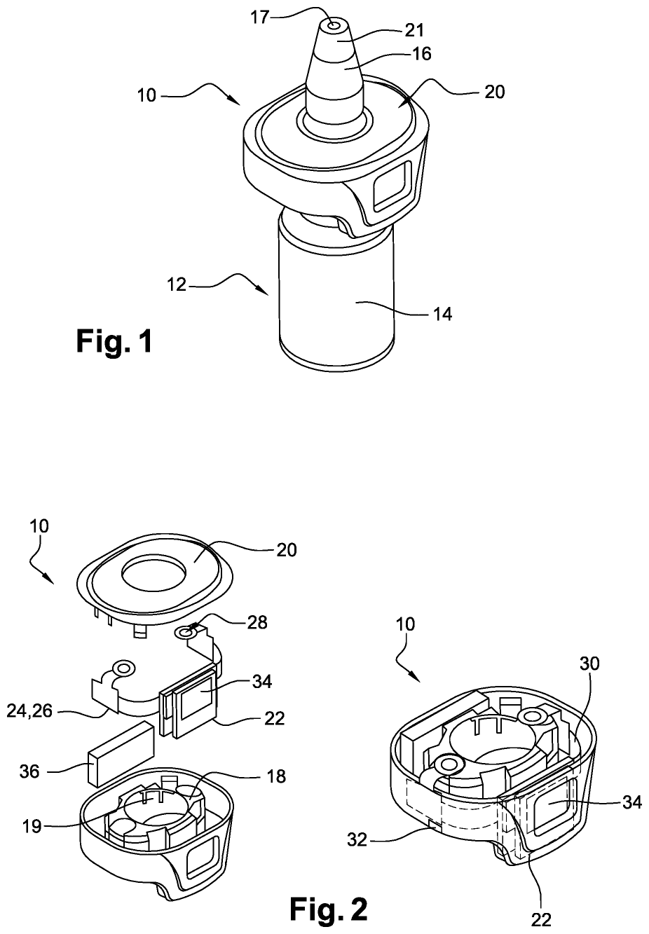

FIG. 1 is a perspective view of an assistance device according to one embodiment connected to a dispensing device,

FIG. 2 is a set of two views of the assistance device of FIG. 1, with on the left a perspective exploded view and on the right a perspective and partially transparent view,

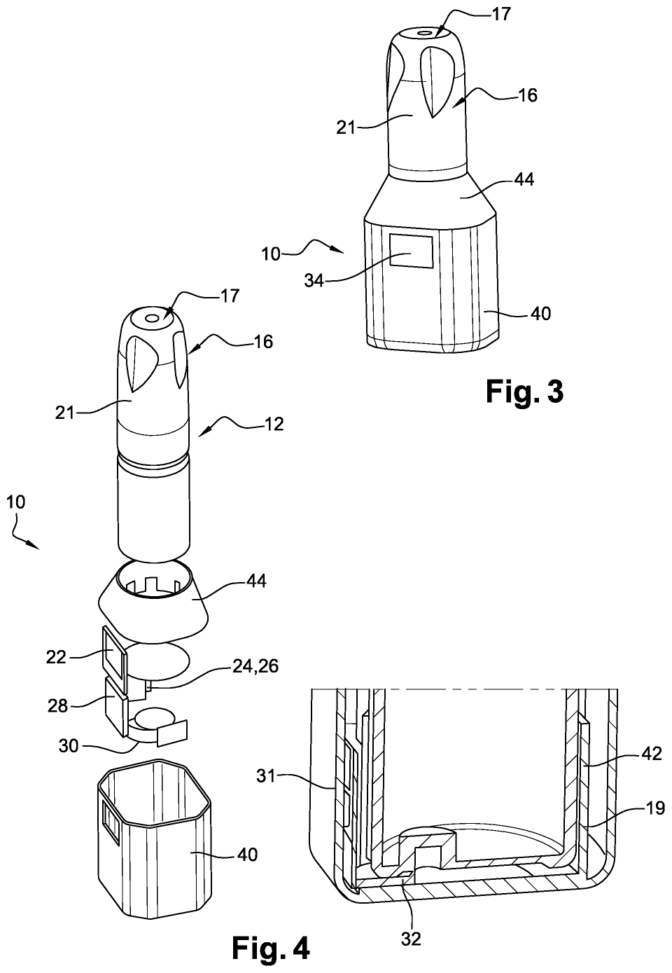

FIG. 3 is a perspective view of an assistance device according to another embodiment connected to a dispensing device,

FIG. 4 is a set of two views of the assistance device of FIG. 3, with on the left a perspective exploded view and on the right a partial longitudinal cross-sectional view including a tank of the dispensing device,

FIG. 5 is a graph showing the steps of a monitoring method according to two different embodiments,

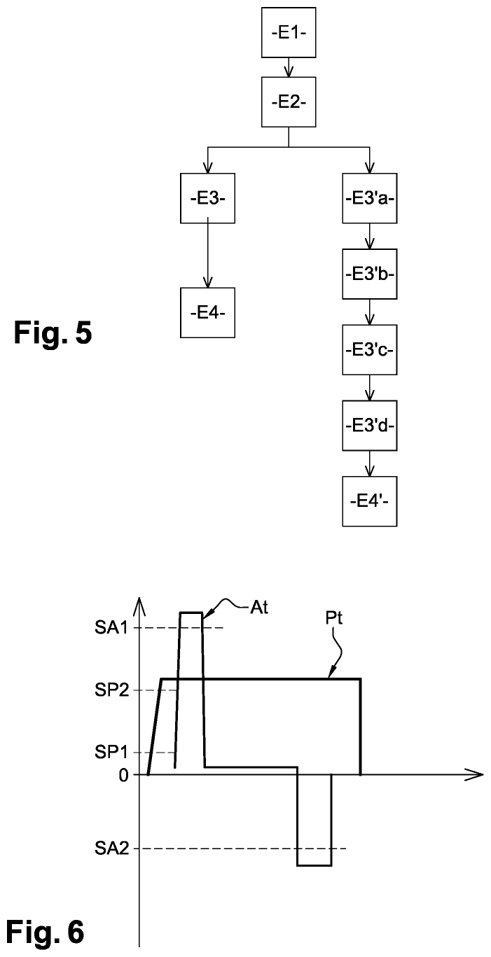

FIG. 6 is a theoretical graph showing physical values quantified and processed by the processing system,

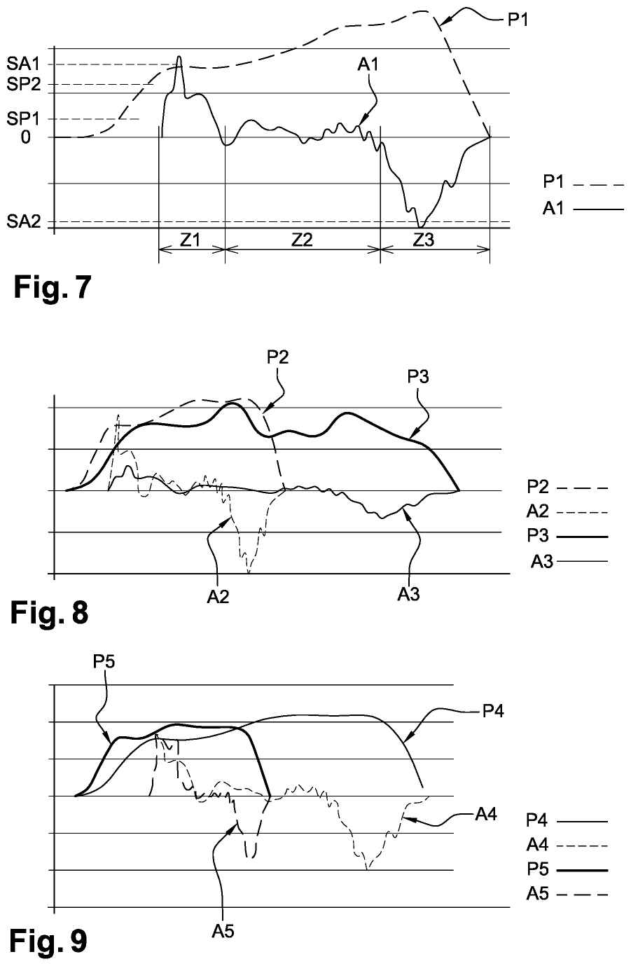

FIGS. 7 to 9 are experimental graphs showing physical values quantified and processed by the processing system.

FIG. 1 illustrates a device 10 for assisting in the use of an axially activated dispensing device 12 for dispensing a product. The dispensing device 12 comprises a tank 14 and a dispensing end piece 16 provided with a nozzle carrying a dispensing orifice 17. The dispensing end piece 16 is attached to the movable part of a pump which comprises a fixed part, fixedly mounted on the tank 14. Thus, the dispensing end piece 16 comprises a gripping area arranged on each side and at the bottom of the nozzle carrying the dispensing orifice 17, in order to activate product dispensing. The assistance device 10 is attached to the dispensing device 12. In this embodiment, it is attached to the dispensing end piece 16. As shown more precisely on FIG. 2, the assistance device 10 takes the form of a hollow annular collar comprising an inner wall forming a neck 18 into which the dispensing end piece 16 is inserted, and a concentric outer wall, forming an outer casing of the assistance device 10. The neck 18 comprises means 19 for connecting to the dispensing device 12, in this example means for clipping the assistance device 10 onto the dispensing end piece 16. As an alternative, or even combined, the means 19 for connecting to the dispensing device 12 comprise means for clipping the assistance device 10 onto the tank 14.

The collar further comprises an upper wall supporting a bearing area 20. The bearing area 20 is mounted so as to be able to move with respect to the tank 14 between a rest position and an activated position in which it activates the dispensing device 12 to obtain the dispensing of product when a user exerts a pressure on the bearing area 20 towards the tank 14 along the dispensing axis. The dispensing axis corresponds to the tank axis, i.e. a vertical axis on FIG. 1. The bearing area 20 comprises two bearing surfaces arranged on each side of the dispensing orifice 17 on which the user places the index finger and the middle finger to exert the axial activation force.

In a particular variant, the dispensing end piece, provided with the dispensing orifice, could form part of the assistance device, which is attached to the tank of the dispensing device when connecting the assistance device 10 and the dispensing device.

The assistance device 10 comprises means 24, 28 for quantifying a physical value linked to the force exerted by the user on the bearing area 20 between the rest position and the activated position, this force being used to move the bearing area 20. It further comprises an information processing system 22 for processing the quantified physical value and providing information on the dispensed product dose. The processing system 22 is a system comprising a set of components (mechanical, electronic, chemical, photonic and/or biological) capable of processing information automatically. It comprises, for example, a printed circuit board (PCB), a set of transistors and/or a computer. The processing system 22 is configured to provide quantitative information on the dispensed product dose, for example a dose volume, a drop size, or a spatial distribution of drops.

The means for quantifying a physical value comprise means 24 for measuring the distance of displacement of the bearing area 20 between the rest position and the activated position. The means 24 for measuring the distance of displacement comprise, in this example, optical means, in particular a transmitter and a receiver of an optical signal, for example an infrared ray. The transmitter and the receiver may both be arranged on a wall of the bearing area 20 facing the tank 14. When the transmitter transmits an optical signal, the signal is reflected on the tank 14 to return to the bearing area 20. It is then picked up by the receiver. The distance of displacement of the bearing area is deduced by measuring the duration between transmission and reception of the optical signal, the speed of the optical signal being known. Alternatively, the means for measuring the distance of displacement can measure the distance of displacement of the tank (or of any element fixedly mounted with respect to the tank) with respect to the bearing area.

The means for quantifying a physical value further comprise means 26 for measuring the duration of displacement of the bearing area 20. The duration of displacement can also be measured by the optical means 24 described above. The information measured by the means 26 for measuring the duration of displacement is then provided to the processing system 22 which deduces information on the speed of displacement of the bearing area 20 with respect to the tank 14, which may correspond to the speed of displacement of the bearing area 20 with respect to any element fixedly mounted with respect to the tank 14, by combining it with the information provided by the means for measuring the distance of displacement, in this case the optical means 24.

Alternatively, the means for measuring the duration of displacement can measure the duration of displacement of the tank (or of any element fixedly mounted with respect to the tank) with respect to the bearing area.

The means for quantifying a physical value also comprise means 28 for measuring the pressure exerted on the bearing area 20, in order to provide information on the magnitude and/or the duration of the pressure exerted by the user on the bearing area 20. In this case, the means 28 for measuring the pressure comprise two Force-Sensing Resistors (FSRs) mounted on a fold of the neck 18 facing the bearing area 20. Each force-sensing resistor 28 is placed at the end of a tab connected to the processing system 22. An example of a suitable force-sensing resistor is that manufactured by Interlink Electronics capable of measuring forces within the range 20 gf to 2.0 kgf (“gf” stands for gram-force, 1 gf=9.80665 mN). Advantageously, the means 28 for measuring the pressure can also act as means for activating and/or waking up any other means for quantifying a physical value when they detect a pressure greater than a predetermined threshold. Alternatively, the means for measuring the pressure can measure the pressure experienced by the tank (or by any element fixedly mounted with respect to the tank) during activation.

The means for quantifying a physical value also comprise means 30 for measuring the acceleration (shown on FIG. 2B) of the bearing area 20 of the assistance device 10. The means 30 for measuring the acceleration comprise an accelerometer fixedly mounted with respect to the bearing area 20. The combination of the information provided by the means 28 for measuring the pressure and the means 30 for measuring the acceleration can be used to determine whether the dispensed dose is complete, while avoiding false positives. Alternatively, the means for measuring the acceleration can measure the acceleration of the tank (or of any element fixedly mounted with respect to the tank) during activation.

The assistance device 10 comprises means 32 for measuring the weight of the dispensing device 12 connected to the assistance device 10, configured to provide information on the amount of product in the tank 14. The means 32 for measuring the weight comprise one or more Force-Sensing Resistors (FSRs) arranged above the tank 14, preferably so as to be able to measure the weight of the dispensing device 12, regardless of its inclination.

The processing system 22 comprises means for reading information shown on the dispensing device 12. The dispensing device 12 comprises an information medium which can be read electronically. This medium is, for example, a QR code (or matrix code), or a radio tag (such as an RFID (radio frequency identification) tag) affixed to the tank 14 or the dispensing end piece 16. The radio tag comprises information such as, in this example, the filling volume of the tank 14, the geometry of the dispensing end piece 16, the product viscosity, the expiry/manufacturing date. The reading means comprise, in this example, an antenna capable of reading the radio tag to extract the information required to process the information.

In a variant, the processing system 22 is connected to an object external to the assistance device 10, for example to a server, a receiver, an intranet or the internet.

The processing system 22 comprises means for storing a variable value on the amount of product remaining in the tank 14, for example a memory integrated in the processing system 22. The assistance device 10 further comprises means 34 for indicating the information provided by the processing system 22, in this case comprising a screen 34 to display information alphanumerically. The assistance device 10 comprises a portable battery 36 for powering the various components, in particular the means for quantifying a physical value and the processing system 22. In a variant, it is supplied with energy by an external power source. Lastly, the assistance device 10 also comprises a temperature sensor near the processing system 22, in order to detect any overheating in the assistance device.

The assistance device may also comprise means for detecting inclination that are coupled to the processing system and are capable of detecting an incorrect inclination of the dispensing device by detecting the inclination of the assistance device. The processing system can be configured to send to the user a signal indicating incorrect use or to indicate to the user how to use the dispensing device correctly.

FIGS. 3 and 4 illustrate an assistance device 10 according to another embodiment. The elements in common with the previous embodiment are identified by the same numerical references.

In this case, the assistance device 10 comprises a main body 40, having an inner seat 42 intended to receive the tank 14 of the dispensing device 12. The inner seat 42 comprises means 19 for connecting to the tank 14. The assistance device 10 further comprises a collar 44 having an orifice through which the dispensing device 12 can be inserted. The dispensing end piece 16 is similar to that of FIG. 1. In this embodiment, the dispensing end piece 16 is provided with finger rests (not shown) acting as a bearing area mounted so as to be able to move with respect to the tank 14, when the user exerts a pressure on the bearing area, between a rest position and an activated product dispensing position. In this case, the dispensing end piece 16 is protected by a cap 21.

The assistance device 10 comprises means 32 for measuring the weight of the dispensing device 12 connected to the assistance device 10, comprising a force-sensing resistor arranged on the inner seat 42 on which the tank 14 rests.

Like the first embodiment, the assistance device 10 also comprises means for quantifying a physical value such as means for measuring the distance 24 and/or the duration 26 of displacement of the bearing area 20, means 28 for measuring the pressure exerted on the bearing area, means 30 for measuring the acceleration of the bearing area, in particular means 30, 31 for measuring the relative acceleration between the bearing area 20 and the connection means 19 or the tank 14, which may comprise two accelerometers, a first accelerometer 30 being fixedly mounted with respect to the bearing area 20 and a second accelerometer 31 being fixedly mounted with respect to the connection means 19, themselves fixedly mounted with respect to the tank 14. According to a variant, the means for measuring the pressure could be replaced by means for measuring the pressure that are connected to the tank, for example by the means for measuring the weight or by other means similar to the means for measuring the pressure and configured to be arranged under the tank, near the means for measuring the weight. Such means for measuring the pressure are means for quantifying a physical value linked to the force exerted by the user on the bearing area, to provide information on the magnitude and/or the duration of the pressure exerted by the user on the bearing area. Advantageously, the means for measuring the pressure can also act as means for activating and/or waking up any other means for quantifying a physical value when they detect a pressure greater than a predetermined threshold.

The assistance device 10 comprises a processing system 22, a display screen 34 and other components similar to that shown on FIGS. 1 and 2.

The assistance device 10 according to the two embodiments can be used to monitor the dispensing of a complete dose during the activation of the dispensing device 12.

FIG. 5 illustrates the steps of an example of monitoring method according to a first embodiment in the left hand branch and according to a second embodiment in the right hand branch, using the assistance device 10 of the preceding figures.

In a first step E1, the means 28 for measuring the pressure, as means for activating and/or waking up the other means, monitor the bearing area 20 to detect a pressure exerted thereon. When the means 28 for measuring the pressure detect a pressure greater than a first predetermined pressure threshold SP1 (shown on FIG. 6), for example a threshold chosen between 2 N and 5 N, they activate and/or wake up the means 30 for measuring the acceleration and trigger the recording of the measurements of pressure and acceleration against time in a second step E2. In the embodiment shown in the left hand branch of the graph, the measurements of pressure and acceleration are recorded for a predetermined duration, for example 0.5 seconds, before being stopped in a step E3. In the next step E4, the means 28 for measuring the pressure and the means 30 for measuring the acceleration send the measurements to the processing system 22 which analyses the profile of the measurements received to provide information on the dispensed dose, for example by comparing it with the theoretical profile of a correct dispensing of FIG. 6, in particular to determine whether or not the dispensed dose was complete.

In the embodiment shown in the right hand part of the graph, the information is processed at the same time as the measurements of pressure and acceleration are taken. In a step E3′a, in fact, the means 28 for measuring the pressure monitor the pressure until it exceeds a second predetermined pressure threshold SP2 (shown on FIG. 6), for example a threshold chosen between 7 N and 10 N. Then, the means 30 for measuring the acceleration monitor the acceleration of the bearing area 20 to observe, over time and in this order, an acceleration greater than a first predetermined acceleration threshold SA1, for example 2 g, during a step E3′b, then a substantially zero acceleration during a step E3′c, and lastly an acceleration less than a second predetermined acceleration threshold SA2, for example −2 g, during a step E3′d. If the test in at least one of the steps E3′a to E3′d is negative, then the processing system 22 deduces that the dispensed dose is not complete. At the end of step E3′d, during a step E4′, the measurements of pressure and acceleration are stopped and the processing system 22 evaluates the duration of the activation, i.e. the duration of steps E3′a to E3′d, or even the duration of each step E3′a to E3′d. If the total duration of steps E3′a to E3′d, or the duration of each step, lies within a predetermined time interval, then the processing system 22 deduces that the dispensed dose is complete.

In both embodiments of the monitoring method, the processing system 22 can for example inform the user on the type of dispensed dose, re-evaluate the number of doses remaining in the tank 14 and/or store the information. It can also process the measurements of pressure and acceleration, preferably in combination with other information such as the weight measurement or the distance measurement and/or the speed of displacement of the bearing area 20, or the measurement of the relative acceleration between the bearing area 20 and the tank 14 (or the means 19 for connecting to the tank 14), to provide quantitative information on the dispensed dose.

FIG. 6 is a theoretical graph showing the measurements of pressure and acceleration of the bearing area 20 over time, if a complete dose is dispensed.

The curve Pt represents the magnitude of the pressure exerted on the bearing area 20 against time. The pressure increases very rapidly at the start of activation, initially exceeding the first predetermined pressure threshold SP1, for example chosen between 2 N and 5 N, from which the components are activated and/or woken up, then the second predetermined pressure threshold SP2, for example chosen between 7 N and 10 N, from which dispensing of product is triggered. The magnitude of the pressure remains above this predetermined pressure threshold SP2 for a duration greater than a predetermined duration threshold, for example 0.5 seconds.

The curve At represents the acceleration of the bearing area 20 against time. It has a crenellated profile and comprises three separate sections. In a first section, once the magnitude of the pressure has exceeded the second predetermined pressure threshold SP2, the acceleration of the bearing area 20 is positive and increases until it exceeds the first predetermined acceleration threshold SA1, for example 2 g. In a second section, the acceleration decreases to become substantially zero for a predetermined period of time, for example 0.2 s. Then, in a third section, the acceleration becomes negative and decreases until it exceeds the second predetermined acceleration threshold SA2, for example −2 g. The acceleration then increases up to zero. Note that the integral of the acceleration over a predefined duration comprises the succession of a positive value (first section of the curve At) and of a negative value (third section of the curve At), each within a predefined time interval.

FIGS. 7 to 9 are three experimental graphs showing the measurements of the pressure exerted on the bearing area 20 and of the acceleration of the bearing area 20 against time.

The graph of FIG. 7 corresponds to the profile of a pressure curve P1 and an acceleration curve A1, corresponding to the dispensing of a complete dose. The pressure curve P1 clearly shows the profile of a pressure which increases and exceeds the two pressure thresholds SP1 and SP2, then which remains above the threshold SP2 for a duration greater than a predetermined duration before decreasing down to zero. The curve A1 clearly shows the three separate zones of the theoretical curve At of FIG. 6. In the first zone Z1 the acceleration is positive, increases and then decreases, the integral of the acceleration is positive, the speed of displacement increases. This first zone Z1 corresponds to the start of activation of the dispensing device 12. In the second zone Z2, the acceleration is substantially zero, the integral of the acceleration is substantially zero, the speed of displacement is constant. This second zone Z2 corresponds to the middle of the activation of the dispensing device 12. In the third zone Z3, the acceleration is negative, decreases then increases, the integral of the acceleration is negative, the speed of displacement decreases. This third zone Z3 corresponds to the end of the activation of the dispensing device 12.

The graph of FIG. 8 corresponds to the profile of two pairs of pressure and acceleration curves (P2, A2) and (P3, A3), the first pair corresponding to the dispensing of a complete dose and the second pair corresponding to the dispensing of an incomplete dose due to an activation duration greater than a predetermined or complete duration but with an incorrect spray in case of spraying (incorrect distribution of drop sizes or incorrect spray profile) for example 1 s, due, for example, to an insufficient pressure over said activation duration.

We see that the profile of the pair of curves (P2, A2) over time corresponds substantially to that of the two theoretical curves of FIG. 6. However, the acceleration curve A3 does not exhibit the three separate sections of the curve At of FIG. 6, in particular it does not initially exceed the first predetermined acceleration threshold SA1 and then does not exceed a second predetermined acceleration threshold SA2. The absence of the two acceleration peaks is due in particular to activation with an insufficient pressure for a duration which is too long, and therefore an activation speed which is too long.

The graph of FIG. 9 corresponds to the profile of two pairs of pressure and acceleration curves (P4, A4) and (P5, A5), the first pair corresponding to the dispensing of a complete dose and the second pair corresponding to the dispensing of an incomplete dose due to an activation duration less than a predetermined duration, for example 0.5 s, due, for example, to a high pressure for an insufficient duration.

We see that the profile of the pair of curves (P4, A4) over time corresponds substantially to that of the two theoretical curves of FIG. 6. However, the profile of the pair of curves (P5, A5) does not correspond to that of the two theoretical curves of FIG. 6. The acceleration curve A5 exhibits, in fact, the three separate sections over a duration less than the predetermined duration. Such an acceleration profile indicates that the user has not exerted a pressure for a duration long enough to move the bearing area from the rest position to the activated position.

The invention is not limited to the embodiments described and other embodiments will be clearly apparent to those skilled in the art. For example, the means for measuring the distance and/or the duration of displacement may comprise other means, such as a Hall effect sensor. This sensor can be fixedly mounted with respect to the bearing area 20 or to the tank 14 and detects the position of either one by the presence of a mechanical part or of a magnet using a known principle.

Furthermore, the dispensing device 12 described herein is a nasal dispensing device, configured to dispense the product as a spray into the nose. The assistance device 10 could be adapted for another type of dispensing device.

1. An assistance device for assisting in a use of an axially activated dispensing device for dispensing a product contained in a tank, a dispensing being activated by a user following a pressure exerted by the user on a bearing area mounted so as to be able to move with respect to the tank between a rest position and an activated product dispensing position, the assistance device comprising:
a connector for connection to the dispensing device,
a measuring device for quantifying a physical value linked to a force exerted by the user on the bearing area between the rest position and the activated position,
an information processing system for processing a quantified physical value and providing information on a dispensed product dose, wherein the measuring device for quantifying a physical value comprises a first measuring device for measuring the pressure exerted by the user on the bearing area in order to provide information on a magnitude or a duration of the pressure exerted by the user on the bearing area or both and a second measuring device for measuring the acceleration of the bearing area.

2. The assistance device according to claim 1, wherein the measuring device for quantifying a physical value further comprises a third measuring device for measuring a distance of displacement of the bearing area between the rest position and the activated position.

3. The assistance device according to claim 2, wherein the measuring device for quantifying a physical value further comprises a fourth measuring device for measuring a duration of displacement of the bearing area, the information processing system being capable of providing information on a speed of displacement of the bearing area with respect to the tank.

4. The assistance device according to claim 3, wherein the third measuring device for measuring the distance of displacement of the bearing area and the fourth measuring device for measuring the duration of displacement of the bearing area comprise an optical device configured to measure the distance of displacement and the duration of displacement, by a transmission and a reception of an optical signal between the bearing area and the tank.

5. The assistance device according to claim 1, wherein the measuring device for quantifying a physical value comprises two accelerometers for measuring a relative acceleration between the bearing area and the tank, a first accelerometer being fixedly mounted with respect to the bearing area and a second accelerometer being fixedly mounted with respect to the tank, the information processing system being capable of processing the acceleration information provided by the two accelerometers in order to deduce the relative acceleration between the bearing area and the tank.

6. The assistance device according to claim 1, further comprising a device for measuring a weight of the dispensing device connected to the assistance device, configured to provide information on an amount of product in the tank.

7. The assistance device according to claim 2, wherein the information processing system is configured to provide quantitative information on the dispensed product dose.

8. The assistance device according to claim 1, wherein the information processing system comprises a device for reading information shown on the dispensing device.

9. The assistance device according to claim 1, wherein the information processing system comprises a storage for storing a variable value corresponding to an amount of product remaining in the tank.

10. The assistance device according to claim 1, further comprising an indicator for indicating the information provided by the information processing system.

11. A method for monitoring a dispensing of a complete dose during an activation of a product dispensing device, by an assistance device according to claim 1, comprising the following steps:
quantifying a physical value linked to a force exerted by a user on the bearing area between a rest position and an activated position,
qualifying a dispensed product dose using the physical value,
wherein the step of quantifying a physical value comprises a step of measuring a pressure exerted by the user on the bearing area and a step of measuring an acceleration of the bearing area.

12. The method according to claim 11, wherein the step of quantifying a physical value comprises a step of measuring a distance of displacement of the bearing area between the rest position and the activated position.

13. The method according to claim 12, wherein the step of quantifying a physical value comprises a step of measuring a duration of displacement of the bearing area between the rest position and the activated position, which is used to deduce a speed of displacement with respect to the tank.

14. The method according to claim 13, wherein the step of measuring the distance of displacement of the bearing area, and the step of measuring the duration of displacement of the bearing area comprises a step of transmitting and a step of receiving an optical signal between the bearing area and the tank.

15. The method according to claim 12, during which, when qualifying the dispensed product dose, the dispensed product dose is considered to be complete when the distance of displacement of the bearing area is greater than a predefined threshold.

16. The method according to claim 11, during which the dispensed product dose is considered to be complete when the magnitude of the pressure exerted by the user exceeds a predefined threshold.

17. The method according to claim 11, wherein the step of quantifying a physical value comprises a step of measuring an acceleration of the bearing area and simultaneously an acceleration of the tank, and a step of determining a relative acceleration of the bearing area with respect to the tank.

18. The method according to claim 17, during which, when qualifying the dispensed product dose, the dispensed product dose is considered to be complete when the relative acceleration measured is greater than a predefined threshold.

19. The method according to claim 11, during which an amount of product dispensed is considered to correspond to a complete dose when an integral of the acceleration of the bearing area for a predefined duration comprises the succession of a positive value and a negative value, each within a predefined time interval.

20. The method according to claim 11, wherein the step of quantifying a physical value comprises a step of measuring a weight of the dispensing device connected to the assistance device.

21. (canceled)

22. (canceled)
